(19) United States
(12) Patent Application Publication
(10) Pub. No.: US 2021/0190699 A1
(43) Pub. Date: Jun. 24, 2021

(54) COLORIMETRIC SENSORS AND METHODS AND USES THEREOF

(71) Applicants: Christopher CAPUTO, Toronto (CA); Ian MALLO, Toronto (CA)

(72) Inventors: Christopher CAPUTO, Toronto (CA); Ian MALLOV, Toronto (CA)

(21) Appl. No.: 17/254,507

(22) PCT Filed: Jul. 5, 2019
(86) PCT No.: PCT/CA2019/050933

§ 371 Date: Dec. 21, 2020

Related U.S. Application Data
(60) Provisional application No. 62/698,458, filed on Jul. 16, 2018.

(51) Int. Cl. G01N 21/78; G01N 31/22; G01N 33/12

(52) U.S. Cl. CPC G01N 21/78; G01N 33/12; G01N 31/223; G01N 31/22

(57) ABSTRACT

A colorimetric sensor for detecting an analyte, wherein the analyte comprises an amine, the sensor comprising genipin, a genipin derivative, a salt thereof, hydrate thereof, solvate thereof, tautomer thereof, optical isomer thereof, or a combination thereof. A container, a system, and/or a device may comprise the colorimetric sensor. The sensor may be used for detecting the presence of the amine, for example, in spoiled food products.

### FIELD

The present invention relates to colorimetric sensors, and methods and uses thereof to determine the presence of an analyte, for example, with respect to indicating the safety of a perishable food product.

### BACKGROUND

Consumers' demand for high-quality food products have led to innovations to maintain food freshness. A wide variety of packaging is available to maintain freshness but how does the consumer determine the condition of the food product? Typically, the packaging of the food product is marked with an expiry date. Unfortunately, the quality and freshness cannot always be determined by the expiry date. Moreover, the expiry date may be illegible or could be missing from the food product. The food product may have been frozen and thawed and its new expiry date difficult to determine. In addition, food spoilage is a massive problem globally, with nearly 25% of all food being discarded because of expiry or the perception that it has expired. There is a need, therefore, for a better way to communicate to the consumer the freshness of the food product.

In terms of food safety and quality issues, sensors can be helpful to communicate to the consumers and provide information about the conditions of the food through a direct visual change. A change in color in response to a change within the product itself (e.g. presence of certain analytes) would be beneficial and easily understood by the consumer. Unfortunately, many sensors available today may not be considered safe (e.g. toxic) in combination with a food product.

Moreover, such sensors may not be limited to the indication of the freshness or safety of a food product. These sensors may also be applicable to workplace safety, for example, in testing for environmental hazards such as toxic analytes.

A need therefore, exists for the development of safer sensors for use with food products to definitively assess the quality thereof, and for a variety of other applications, including the detection of possible environmental hazards.

### SUMMARY

In accordance with an aspect, there is provided a colorimetric sensor for detecting an analyte, wherein the analyte comprises an amine, the sensor comprising genipin, a genipin derivative, a salt thereof, hydrate thereof, solvate thereof, tautomer thereof, optical isomer thereof, or a combination thereof.

In another aspect, wherein there is a visible color change. In another aspect, wherein the amine comprises a primary amine. In another aspect, wherein the amine is selected from ammonia, biogenic amines, amino acids, amides, polypeptides, proteins, substituted and unsubstituted alkyl amines, substituted and unsubstituted aromatic amines, substituted and unsubstituted heteroaromatic amines, or a combination thereof. In another aspect, wherein the amine is selected from benzyl amine, 4-(2-aminoethyl)phenol, N-methylbenzylamine, N,N-dimethylbenzylamine, 1,4 diaminobutane, 1,5-diaminopentane, or a combination thereof. In another aspect, wherein the amine is selected from 4-(2-aminoethyl)phenol, 1,4 diaminobutane, 1,5-diaminopentane, or a combination thereof. In another aspect, wherein the amine is vaporous. In another aspect, wherein the amine has a high vapor pressure. In another aspect, wherein the amine is a gaseous amine. In another aspect, wherein the genipin, the genipin derivative, the salt thereof, hydrate thereof, solvate thereof, tautomer thereof, optical isomer thereof, or the combination thereof undergoes a visible color change upon exposure to the amine. In another aspect, wherein the genipin undergoes a visible color change upon exposure to the amine. In another aspect, wherein the genipin derivative undergoes a visible color change upon exposure to the amine. In another aspect, wherein the genipin derivative is:

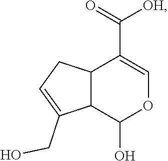 [SMILES: O=C(O)C1=COC(O)C2C(CO)=CCC12]

a salt thereof, hydrate thereof, solvate thereof, tautomer thereof, optical isomer thereof, or a combination thereof.

In another aspect, wherein the visible color change is a color that absorbs in a wavelength range of about 375 nm to about 490 nm. In another aspect, wherein the visible color change is a reddish color. In another aspect, wherein the sensor comprises genipin. In another aspect, wherein the analyte further comprises an oxidizing agent. In another aspect, wherein the oxidizing agent is selected from oxygen, ozone, nitric oxide, nitrous oxide, hydrogen peroxide, or a combination thereof. In another aspect, wherein the oxidizing agent is oxygen. In another aspect, wherein the genipin, the genipin derivative, the salt thereof, hydrate thereof, solvate thereof, tautomer thereof, optical isomer thereof, or the combination thereof undergoes a further visible color change upon exposure to the oxidizing agent. In another aspect, wherein the genipin undergoes a further visible color change upon exposure to the oxidizing agent. In another aspect, wherein the genipin derivative undergoes a further visible color change upon exposure to the oxidizing agent. In another aspect, wherein the visible color change is a color that absorbs in a wavelength range of about 550 nm to about 650 nm. In another aspect, wherein the visible color change is a blue/violet color.

In another aspect, the sensor further comprises a solvent. In another aspect, wherein the solvent is selected whereby the genipin, the genipin derivative, the salt thereof, hydrate thereof, solvate thereof, tautomer thereof, optical isomer thereof, or the combination thereof is sufficiently soluble such that the colorimetric sensor is capable of changing color in the presence of the amine. In another aspect, wherein the solvent is selected from water, alcohols, or a combination thereof. In another aspect, wherein the solvent is a vegetable oil, a natural oil, an essential oil, or a combination thereof. In another aspect, the sensor further comprising a thickening agent. In another aspect, wherein the sensor is a gel. In another aspect, wherein the sensor is multiple particulates. In another aspect, wherein the sensor is selected from powder, granules, tablets, pellets, beads, mini-tabs, spherules, beadlets, microcapsules, milli-spheres, nano-capsules, micro-spheres, capsules, or a combination thereof. In another aspect, wherein the capsules comprises a semi-permeable coating or semi-permeable encapsulation.

In another aspect, the sensor further comprises a substrate. In another aspect, wherein the genipin, the genipin derivative, the salt thereof, hydrate thereof, solvate thereof, tautomer thereof, optical isomer thereof, or the combination thereof is in the form of a coating on the substrate, intermixed with the substrate to form a matrix, impregnated in the substrate, at least a portion covalently bound to the substrate, non-covalently bound to the substrate, or a combination thereof. In another aspect, wherein the genipin, the genipin derivative, the salt thereof, hydrate thereof, solvate thereof, tautomer thereof, optical isomer thereof, or the combination thereof is non-covalently bound to the substrate. In another aspect, wherein the substrate is selected from a woven or non-woven material, a polymer, or a combination thereof. In another aspect, wherein the substrate is selected from paper, cotton, fabric, sponge, foam, glass and ceramic fibre paper, nylon, rayon, a matrix, bead, a film-forming or fiber-forming polymer, or a combination thereof. In another aspect, wherein the substrate is polymeric. In another aspect, wherein the substrate is polymeric packaging. In another aspect, wherein the substrate is a gel substrate. In another aspect, wherein the gel substrate is bead(s) or a film. In another aspect, wherein the gel substrate comprises a carbohydrate polymer. In another aspect, wherein the carbohydrate polymer comprises an anionic polysaccharide. In another aspect, wherein the carbohydrate polymer comprises an alkali and/or alkaline earth metal alginate. In another aspect, wherein the sensor comprises beads, wherein the beads comprise genipin and the alginate. In another aspect, wherein the beads are in a container such as a pouch, sachet or vial that is permeable or semi-permeable.

In another aspect, the sensor further comprises an additive to control the color change of genipin, a genipin derivative, a salt thereof, hydrate thereof, solvate thereof, tautomer thereof, optical isomer thereof, or a combination thereof. In another aspect, wherein the additive is a regulator that inhibits or enhances the reaction of the analyte with genipin, a genipin derivative, a salt thereof, hydrate thereof, solvate thereof, tautomer thereof, optical isomer thereof, or a combination thereof.

In accordance with another aspect, there is provided a container comprising the colorimetric sensor described herein. In another aspect, wherein the container is selected from a pouch, sachet or vial. In another aspect, wherein the container is permeable or semi-permeable.

In accordance with another aspect, there is provided a system comprising the colorimetric sensor described herein.

In accordance with yet another aspect, there is provided device comprising the colorimetric sensor described herein.

In accordance with another aspect, there is provided use of the sensor described herein for detecting the presence of the amine. In accordance with another aspect, there is provided a method for detecting the presence of the amine, the method comprising exposing the sensor of described herein to the amine. In another aspect, wherein the amine is released from food spoilage. In another aspect, wherein the amine is released in an industrial facility.

It is understood that one or more of the aspects described herein (and above) may be combined in any suitable manner. The novel features of the present invention will become apparent to those of skill in the art upon examination of the following detailed description of the invention. It should be understood, however, that the detailed description of the invention and the specific examples presented, while indicating certain aspects of the present invention, are provided for illustration purposes only because various changes and modifications within the spirit and scope of the invention will become apparent to those of skill in the art from the detailed description of the invention and claims that follow.

### BRIEF DESCRIPTION OF THE DRAWINGS

The present invention will be further understood from the following description with reference to the Figures, in which:

FIG. 1 shows UV-Vis Absorbance Spectrum of a methanol solution of 1,4-diaminobutane (2.0×10⁻³ M).

FIG. 2 shows UV-Vis Absorbance Spectrum of a methanol solution of Genipin (2.0×10⁻³ M).

FIG. 3 shows UV-Vis Absorbance Spectrum of a methanol solution of Genipin (8.0×10⁻⁵ M).

FIG. 4 shows UV-Vis Absorbance Spectrum of individual reactions of benzylamine (2.0×10⁻³ M), N-methylbenzylamine (2.0×10⁻³ M), or N,N-dimethylbenzylamine (2.0×10⁻³ M) with Genipin (2.0×10⁻³ M) in methanol after 48 h.

FIG. 5 shows UV-Vis Absorbance Spectra of a reaction mixture of 1,4-diaminobutane (Putrescine) (2.0×10⁻³ M) with Genipin (2.0×10⁻³ M) in 50 ml methanol and monitored over 1-120 h.

FIG. 6 shows UV-Vis Absorbance Spectra of a reaction mixture of 1,4-diaminobutane (2.0×10⁻³ M) with Genipin (2.0×10⁻³ M) in methanol after 24 h in the presence and absence of oxygen.

FIG. 7 shows UV-Vis Absorbance Spectra of a reaction mixture of 1,4-diaminobutane (2.0×10⁻³ M) with Genipin (2.0×10⁻³ M) in methanol after 48 h in the presence and absence of oxygen.

FIG. 8 shows UV-Vis Absorbance Spectra of a reaction mixture of 1,4-diaminobutane (2.0×10⁻³ M) with Genipin (2.0×10⁻³ M) in methanol over 24-48 h in the presence and absence of oxygen.

FIG. 9 shows UV-Vis Absorbance Spectra of a reaction mixture of Genipin (0.20M) and 1,4-diaminobutane (0.20 M) in methanol after 4 h and 7 h reaction times and diluted at time of measurement to 2.0×10⁻³M.

FIG. 10 shows UV-Vis Absorbance Spectra of a reaction mixture of 2.0×10⁻³ M Genipin and 1,4-diaminobutane in methanol after 48 h. Samples were diluted to various concentrations and measured.

FIG. 11 shows UV-Visible Absorbances at λₘₐₓ=605 nm for varying sample concentrations adjusted from a reaction mixture of Genipin (2.0×10⁻³ M) and 1,4-diaminobutane (2.0×10⁻³ M) in methanol after 48 h.

FIG. 12 shows comparison of UV-Vis Absorbance Spectra of i) a reaction mixture of Genipin (0.20 M) and 1,4-diaminobutane (0.20 M) in methanol after 48 h (diluted at time of measurement to 2.0×10⁻³M) and ii) a reaction mixture of Genipin (2.0×10⁻³ M) and 1,4-diaminobutane (2.0×10⁻³ M) in methanol after 48 h.

FIG. 13 shows comparison of UV-Vis Absorbance Spectra of reaction mixtures of Genipin (2.0×10⁻³ M) and 1,5-diaminopentane (Cadaverine) (2.0×10⁻³ M) in methanol over 1-48 h.

FIG. 14 shows comparison of UV-Vis Absorbance Spectra of reaction mixtures of Genipin (2.0×10⁻³ M) and 1,5-diaminopentane (2.0×10⁻³ M) in methanol over 1-48 h (sample diluted 5× at time of measurement).

FIG. 15 shows comparison of UV-Vis Absorbance Spectra of reaction mixtures of Genipin (2.0×10⁻³ M) and 1,5-diaminopentane (2.0×10⁻³ M) in methanol over 1-48 h. Samples were diluted to various concentrations and measured.

FIG. 16 shows Absorbance vs Concentration graph for Genipin and 1,5-diaminopentane in methanol after 48 h.

FIG. 17 shows comparison of UV-Vis Absorbance Spectra of reaction mixtures of Genipin (2.0×10⁻³ M) and 4-(2-aminoethyl)phenol (Tyramine) (2.0×10⁻³ M) in methanol over 1-48 h.

FIG. 18 shows comparison of UV-Vis Absorbance Spectra of reaction mixtures of Genipin (2.0×10⁻³ M) and 4-(2-aminoethyl)phenol (2.0×10⁻³ M) in methanol over 1-48 h (sample diluted 10× at time of measurement).

FIG. 19 shows comparison of UV-Vis Absorbance Spectra of reaction mixtures of Genipin (2.0×10⁻³ M) and 4-(2-aminoethyl)phenol (2.0×10⁻³ M) in methanol.

FIG. 20 shows Absorbance vs Concentration graph for Genipin and 4-(2-aminoethyl)phenol in methanol after 48 h.

FIG. 21 shows comparison of UV-Vis Absorbance Spectra of reaction mixtures of various biogenic amines and Genipin in methanol (samples measured at indicated dilutions).

FIGS. 22A-C show FIG. 22A) gel beads at t=0 with 1,4-diaminobutane vapor; FIG. 22B) gel beads after 24 hours exposure to 1,4-diaminobutane vapor; FIG. 22C) comparison of gel beads exposed to 1,4-diaminobutane vapor after 48 hours at room temperature (left vial) and in the refrigerator (right vial).

FIG. 23 shows UV-Vis Absorbance Spectrum of a solution of Genipin beads measured after reaction with 1,4-diaminobutane for 48 h; the Genipin beads were dissolved in 3.5 mL of a 0.055 M solution of sodium citrate (about 3.5 mM of Genipin).

FIG. 24 shows Genipin beads placed next to raw chicken after 2 hours at room temperature (left) and two hours in the refrigerator at 4° C. (right).

### DETAILED DESCRIPTION OF CERTAIN ASPECTS

### Definitions

Unless otherwise explained, all technical and scientific terms used herein have the same meaning as commonly understood by one of ordinary skill in the art to which this disclosure belongs. Although any methods and materials similar or equivalent to those described herein can be used in the practice for testing of the present invention, the typical materials and methods are described herein. In describing and claiming the present invention, the following terminology will be used.

It is also to be understood that the terminology used herein is for the purpose of describing particular aspects only and is not intended to be limiting. Patent applications, patents, and publications are cited herein to assist in understanding the aspects described. All such references cited herein are incorporated herein by reference in their entirety and for all purposes to the same extent as if each individual publication or patent or patent application was specifically and individually indicated to be incorporated by reference in its entirety for all purposes. To the extent publications and patents or patent applications incorporated by reference contradict the disclosure contained in the specification, the specification is intended to supersede and/or take precedence over any such contradictory material.

In understanding the scope of the present application, the articles “a”, “an”, “the”, and “said” are intended to mean that there are one or more of the elements. Additionally, the term “comprising” and its derivatives, as used herein, are intended to be open ended terms that specify the presence of the stated features, elements, components, groups, integers, and/or steps, but do not exclude the presence of other unstated features, elements, components, groups, integers and/or steps. The foregoing also applies to words having similar meanings such as the terms, “including”, “having” and their derivatives.

It will be understood that any aspects described as “comprising” certain components may also “consist of” or “consist essentially of,” wherein “consisting of” has a closed-ended or restrictive meaning and “consisting essentially of” means including the components specified but excluding other components except for materials present as impurities, unavoidable materials present as a result of processes used to provide the components, and components added for a purpose other than achieving the technical effect of the invention. For example, a composition defined using the phrase “consisting essentially of” encompasses any known acceptable additive, excipient, diluent, carrier, and the like. Typically, a composition consisting essentially of a set of components will comprise less than 5% by weight, typically less than 3% by weight, more typically less than 1%, and even more typically less than 0.1% by weight of non-specified component(s).

It will be understood that any component defined herein as being included may be explicitly excluded from the claimed invention by way of proviso or negative limitation.

In addition, all ranges given herein include the end of the ranges and also any intermediate range points, whether explicitly stated or not.

Terms of degree such as “substantially”, “about” and “approximately” as used herein mean a reasonable amount of deviation of the modified term such that the end result is not significantly changed. These terms of degree should be construed as including a deviation of at least ±5% of the modified term if this deviation would not negate the meaning of the word it modifies.

The abbreviation, “e.g.” is derived from the Latin exempli gratia, and is used herein to indicate a non-limiting example. Thus, the abbreviation “e.g.” is synonymous with the term “for example.” The word “or” is intended to include “and” unless the context clearly indicates otherwise.

It is further to be understood that all molecular weight or molecular mass values, are approximate and are provided for description. Although methods and materials similar or equivalent to those described herein can be used in the practice or testing of this disclosure, suitable methods and materials are described below.

As used herein, the term polymer means a chemical compound or mixture of compounds formed by polymerization and consisting essentially of repeating structural units.

As used herein, a dimer or a dimeric compound means a compound consisting essentially of two monomers.

As used herein, a trimer or a trimeric compound means a compound consisting essentially of three monomers.

The term tautomer or tautomeric isomer as used herein means compounds that can be interconvertible through tautomerization (e.g. keto-enol tautomerization).

As used herein the term amine or amine compound is understood to include, for example, ammonia, biogenic amines, amino acids, amides, polypeptides, proteins, substituted and unsubstituted alkyl amines, substituted and unsubstituted aromatic amines, and substituted and unsubstituted heteroaromatic amines. Such amines encompass, for example, primary, secondary, and tertiary amines. Examples include, benzyl amine, N-methylbenzylamine, N,N-dimethylbenzylamine, 1,4 diaminobutane, 1,5-diaminopentane, and 4-(2-aminoethyl)phenol.

The term “genipin derivative” generally refers to a compound that results from a modification to genipin but the compound maintains the chemical/physical property that a visible color change occurs upon exposure to at least an amine. For example, visible color change occurs upon exposure to at least a primary amine. In another example, visible color change occurs upon exposure to at least an amine and oxidizing agent. In another example, visible color change occurs upon exposure to at least an amine and oxygen. More typically, visible color change occurs upon exposure to at least a primary amine and oxygen. For example, other iridoid compounds may be used with the chemical/physical property that a visible color change occurs upon exposure to at least an amine. For example, the methyl ester of genipin may be converted to a carboxylic acid derivative:

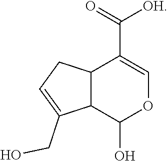 [SMILES: O=C(O)C1=COC(O)C2C(CO)=CCC12]

Although a statement such as “genipin, a genipin derivative, a salt thereof, hydrate thereof, solvate thereof, tautomer thereof, optical isomer thereof, or a combination thereof” is used, it is understood that this statement includes support for each individual component.

The term “oxidizing agent” is given its ordinary meaning in the art and generally refers to a reactant that has the ability to oxidize or increase the oxidation state of another reactant. Non-limiting examples of oxidizing agents include oxygen (O₂), ozone (O₃) nitric oxide, nitrous oxide, and hydrogen peroxide. In some embodiments, the oxidizing agent is oxygen. Alternative or additional oxidizing agents or a combination of oxidizing agents may be used, in some embodiments.

The term “substituted” refers to moieties having one, two, three or more substituents, which may be the same or different, each replacing a hydrogen atom. Examples of substituents include but are not limited to halogen (e.g., F, Cl, Br, and I), hydroxyl, amino, carboxy, cyano, alkoxy, alkyl, aryl, aralkyl, acyloxy, nitro, and haloalkyl.

As used herein, the term “salt” is understood to include suitable salts of the compounds described herein (e.g. salts of Genipin). Examples of salts include salts of the compound having a cation as a counterion, such as an alkaline metal ion (e.g., Na⁺, K⁺, etc.), an alkaline earth metal ion (e.g., Mg²⁺, Ca²⁺, etc.), ammonium ion (e.g., NH₄⁺ or an organic ammonium ion), etc.

The salts of the compounds described herein can be synthesized by conventional chemical methods from the compounds described herein.

### Genipin and Derivatives Thereof

Genipin is a colorless, natural product.

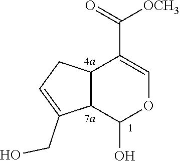 [SMILES: COC(=O)C1=COC(O)C2C(CO)=CCC12]

Genipin is a hydrolytic product of geniposide, which is found in the fruit of Gardenia jasminoides Ellis, and is biodegradable, has low cytotoxicity, and has been used as a cross-linking agent. It has been used in various food, cosmetic and drugs as a blue dye. Genipin reacts with amines to change from colourless to a yellow intermediate to a red (or reddish brown) intermediate and, in the further presence of an oxidizing agent, such as oxygen, it changes to a blue product. The red intermediate is stable in the absence of an oxidizing agent, such as oxygen.

In embodiments, genipin has the ability to act as a colorimetric sensor for an analyte, in particular, when the analyte is an amine. In other embodiments, genipin has the ability to act as an amine colorimetric sensor. In certain embodiments, genipin has the dual ability to act as an amine and oxidizing agent colorimetric sensor. In particular, genipin has the dual ability to act as an amine and oxygen colorimetric sensor. More specifically, genipin acts as a primary amine colorimetric sensor or a primary amine and oxygen colorimetric sensor.

With respect to the embodiments described herein, genipin, a salt thereof, hydrate thereof, solvate thereof, tautomer thereof, optical isomer thereof, or a combination thereof, may be used as a colorimetric sensor described herein. As denoted by genipin's structure, there are three stereocenters. In certain embodiments, genipin can exist as SRR (1S,4aR,7aR) or its enantiomer RSS (1R,4aS,7aS).

With respect to further embodiments described herein, a genipin derivative, a salt thereof, hydrate thereof, solvate thereof, tautomer thereof, optical isomer thereof, or a combination thereof, may be used as a colorimetric sensor described herein.

With respect to further embodiments described herein, genipin, a genipin derivative, a salt thereof, hydrate thereof, solvate thereof, tautomer thereof, optical isomer thereof, or a combination thereof, may be used as a colorimetric sensor described herein.

With respect to genipin derivatives, examples may include other iridoid compounds with the chemical/physical property that a visible color change occurs upon exposure to at least an amine and more specifically, a primary amine. In other examples of derivatives, the methyl ester may be converted to a carboxylic acid derivative. In addition, the genipin derivative may be a dimer, trimer or polymer of genipin.

### Substrates for Genipin and Derivatives Thereof

In an embodiment, the colorimetric sensor comprises genipin, a genipin derivative, a salt thereof, hydrate thereof, solvate thereof, tautomer thereof, optical isomer thereof, or a combination thereof.

In another embodiment, the colorimetric sensor comprises genipin, a genipin derivative, a salt thereof, hydrate thereof, solvate thereof, tautomer thereof, optical isomer thereof, or a combination thereof, and a solvent. The solvent may be any suitable solvent. For example, water, alcohols (e.g. methanol, ethanol, isopropanol, etc.), or a combination thereof. In certain embodiments, the solvent can be any solvent that provides suitable solubility of the genipin, a genipin derivative, a salt thereof, hydrate thereof, solvate thereof, tautomer thereof, optical isomer thereof, or a combination thereof such that the colorimetric sensor is capable of changing color in the presence of an amine.

The solvent may also be an oil, such as, and without being limited thereto, suspensions or emulsions. The oil may include, for example, vegetable oil, any natural or essential oil, including but not limited to oils from canola, rosemary, olive, coconut, corn, cottonseed, palm, peanut, safflower, sesame, soybean, sunflower, almond, cashew, hazelnut, macadamia, pecan, pistachio, walnut, acai and others. In other embodiments, glycerol is used in place of vegetable oil or long chain fatty acids. Typically, alternatives to the vegetable oil are non-toxic, organic, non-amine-containing liquid compounds. The oils may assist in forming gels.

In another embodiment, the colorimetric sensor may further comprise a thickening agent. In particular embodiments, the thickening agent is used to form a gel or paste. Any suitable thickening agent may be used, for example, to absorb water and increase the viscosity of the colorimetric sensor. Examples include xanthan gum, natural gums such as alginin, locust bean, guar, acacia, oat, karaya, tara, gellan, ghatti, konjac, cassia, tragacanth, arabinogalactan, carob, spruce, chicle, dammar and curdlan. Other polysaccharides that may be used as thickeners include pectin, carrageenan, pullulan, baker's yeast glycan, and soybean hemicellulose.

In other embodiments, the colorimetric sensor may be formulated into multiple particulates such as a powder or granules. In further embodiments, the colorimetric sensor powder/granules may be formulated into a tablet using any suitable techniques known to those skilled in the formulation of tablets. For example, the genipin, a genipin derivative, a salt thereof, hydrate thereof, solvate thereof, tautomer thereof, optical isomer thereof, or the combination thereof may be milled with suitable binders and pressed to form a tablet. Examples of binders include crystalline cellulose, white sugar, D-mannitol, dextrin, hydroxypropylcellulose, hydroxypropylmethyl-cellulose, polyvinylpyrrolidone, starch, sucrose, gelatin, methylcellulose, carboxymethylcellulose sodium and the like. In additional embodiments, pellets, beads, mini-tabs, spherules, beadlets, microcapsules, milli-spheres, nano-capsules, micro-spheres, or capsules may be used.

In embodiments, the colorimetric sensor comprises genipin, a genipin derivative, a salt thereof, hydrate thereof, solvate thereof, tautomer thereof, optical isomer thereof, or a combination thereof, and a substrate. A substrate may be any suitable material for genipin, a genipin derivative, a salt thereof, hydrate thereof, solvate thereof, tautomer thereof, optical isomer thereof, or a combination thereof such that the substrate does not substantially affect the function of the sensor. The substrate may be any suitable shape.

Some examples include genipin, a genipin derivative, a salt thereof, hydrate thereof, solvate thereof, tautomer thereof, optical isomer thereof, or a combination thereof, being used as a coating on a substrate, intermixed with a substrate to form a matrix, impregnating a substrate (e.g. porous substrate), at least a portion covalently bound to a substrate, and/or non-covalently bound to a substrate. Some examples of substrates include woven or non-woven material, such as paper, cotton, fabric, sponge, foam, glass and ceramic fibre paper, nylon, rayon, etc.; and a polymer, such as a matrix, bead, a film-forming or fiber-forming polymer.

Such substrates may include multiple layers. The composition of the genipin, a genipin derivative, a salt thereof, hydrate thereof, solvate thereof, tautomer thereof, optical isomer thereof, or a combination thereof, may be absorbed, adsorbed, coated, intermixed, at least a portion covalently bound, non-covalently bound to a substrate, and/or impregnated on/to the substrate. The substrate may be polymeric (e.g. plastic packaging). The substrate may be a food packaging. A composition of the genipin, a genipin derivative, a salt thereof, hydrate thereof, solvate thereof, tautomer thereof, optical isomer thereof, or a combination thereof may be absorbed, adsorbed, coated, intermixed, covalently bound and/or impregnated on/to the interior of the food packaging. In further examples, the composition can be used as a cross-linker to form polymeric packaging therefrom. Another example, includes filter paper, whereby the composition is absorbed onto the filter paper using a solution of the composition. A further example, includes pellets, beads, mini-tabs, spherules, beadlets, microcapsules, milli-spheres, nano-capsules, micro-spheres, or capsules of the composition. These formulations may comprise the composition with the coating or encapsulation being semi-permeable. The formulations may be placed in a container such as a pouch, sachet or vial that is permeable or semi-permeable.

In specific embodiments, the substrate is a gel substrate. It is understood that the gel substrate can be any suitable shape for functioning as a sensor. The gel substrate can be, for example, bead(s) or a film. In an example, the gel is formulated by combining genipin, a genipin derivative, a salt thereof, hydrate thereof, solvate thereof, tautomer thereof, optical isomer thereof, or a combination thereof and a suitable polymer such as, and without being limited thereto, a carbohydrate polymer, more specifically, an anionic polysaccharide or other polymer gel. In specific embodiments, the polymer is an alkali and/or alkaline earth metal alginate, such as calcium or sodium alginate and forming, for example, beads and/or gels. Typically, alkaline earth metal alginate may form discrete beads and gels may be formed from alkali metal alginate. The beads and/or gels may be placed in a container such as a pouch, sachet or vial that is permeable or semi-permeable. Using a fruit extract (e.g. genipin) and a polymeric material based on algae, this embodiment is an edible product, therefore, regulatory barriers to implementation will be lower.

The dimensions of the substrate can be any suitable dimension. In embodiments, the dimensions are such that a desired ratio of surface area to volume and application requirements i.e. visibility, packaging, mechanical reading means, etc. is achieved.

In additional embodiments, the sensor may also comprise an additive to further control the color change of genipin, a genipin derivative, a salt thereof, hydrate thereof, solvate thereof, tautomer thereof, optical isomer thereof, or a combination thereof. For example, the additive may act as a regulator to inhibit or enhance the reaction of the analyte with genipin, a genipin derivative, a salt thereof, hydrate thereof, solvate thereof, tautomer thereof, optical isomer thereof, or a combination thereof, depending on the application needed. For example, additives may include, but are not limited to, alcohols, surfactants, or catalysts. In more specific examples, additives containing carbonyl groups capable of reacting with amines may be used. Additives may be used such that when the concentration of the amine is at a first threshold value, the additive reacts with the amine, and when the concentration of the amine is at a second threshold value, the genipin, a genipin derivative, a salt thereof, hydrate thereof, solvate thereof, tautomer thereof, optical isomer thereof, or a combination thereof reacts with the amine indicating the second threshold has been reached. The second threshold may be, for example, that the amine level has reached a hazardous level.

### Concentrations

Any suitable concentration of the genipin, a genipin derivative, a salt thereof, hydrate thereof, solvate thereof, tautomer thereof, optical isomer thereof, or a combination thereof may be used. One skilled in the art can determine the concentration to achieve the sensitivity needed for a certain application (e.g. food product, environmental hazard, etc.). One skilled in the art, for example, can take into account the exposure time, temperature, and/or surface area to volume ratio to determine the concentration needed for a certain application (e.g. food product, environmental hazard, etc.).

In some examples, concentrations of genipin in gel beads may be about 4 to about 40 mM in the gel solution used to make the beads, or about 3 to about 30 ng of genipin per bead, with bead masses in the range of about 10 to about 50 mg. These ranges, as noted above, include the end of the ranges and also any intermediate range points, whether explicitly stated or not.

### Applications of the Colorimetric Sensor

The colorimetric sensor described herein may be used in a variety of areas where a visible detection indicating the presence of an analyte is desirable.

Examples of the colorimetric sensor described herein may be used to detect the presence of amines. Amines, in particular, biogenic amines, are produced as food spoils. Examples of biogenic amines include putrescine, cadaverine and tyramine. These are typical amines released from spoiled food products (Onal, A., Food Chemistry, 2007, 103, 1475-1486). Foods such as meat, fish, mushrooms and wines release such amines as each spoil. As mentioned, food spoilage is a massive problem globally, and the colorimetric sensor described herein can assist with determining the food quality, and since best-before dates are arbitrary, this may provide better assessment of the food quality.

The colorimetric sensor described herein interacts with an analyte to produce a visually discernible color change. In embodiments, the colorimetric sensor will change color and absorb in a wavelength range of about 350 nm to about 560 nm, typically, a wavelength range of about 375 nm to about 560 nm, about 375 nm to about 500 nm, about 375 nm to about 500 nm, or about 375 nm to about 490 nm in the presence of an amine. It is more typically a reddish color (e.g. absorbing wavelengths of about 375 nm to about 490 nm) in the presence of an amine. In the further presence of an oxidizing agent, such as oxygen, it will change color and absorb in a wavelength range of about 550 nm to about 750 nm, typically, a wavelength range of about 550 nm to about 650 nm, and more typically, about 590 nm to about 630 nm. It is more typically a blue color (e.g. absorbing wavelengths of about 590 nm to about 630 nm). In certain embodiments, the red color will not convert to blue if an oxidizing agent, such as oxygen, is not present. The colorimetric sensor may be a dual colorimetric sensor to act as an amine and oxidizing agent colorimetric sensor to determine food spoilage. In particular, the sensor has the dual ability to act as an amine and oxygen colorimetric sensor. More specifically, the sensor acts as a primary amine and oxygen colorimetric sensor.

In embodiments, the colorimetric sensor described herein can measure amines with a high vapor pressure. In other words, the vapor is a mixture of two phases: liquid and gas at room temperature. In another embodiment, the colorimetric sensor described herein can measure gaseous amines.

Applications of the colorimetric sensor described herein may not be limited to food quality. Amine sensing has applications in manufacturing, such as fish canning plants or other industrial work areas, and/or in environmental applications, where amines can pose a major health issue. Therefore, applications include workplace safety and environmental contamination.

The colorimetric sensor described herein can simply be exposed to an amine vapor and/or gases to detect the presence of such an analyte. For example, the sensor may be placed with the food within its food packaging or placed within an environment where amines are released.

The colorimetric sensor may be included in a device or system for testing an analyte.

The above disclosure generally describes the present invention. A more complete understanding can be obtained by reference to the following specific examples. These examples are provided for purposes of illustration only and are not intended to be limiting unless otherwise specified. Thus, the invention should in no way be construed as being limited to the following examples, but rather, should be construed to encompass any and all variations which become evident as a result of the teaching provided herein.

Without further description, it is believed that one of ordinary skill in the art can, using the preceding description and the following illustrative examples, make and utilize the compounds of the present invention and practice the claimed methods. The following working examples therefore, specifically point out the typical aspects, and are not to be construed as limiting in any way the remainder of the disclosure.

### EXAMPLES

All UV-visible spectra were recorded using quartz cuvettes on an Agilent Cary-5000 UV-vis-NIR spectrophotometer over wavelength ranges of 200-800 nm. Solvents and reagents were purchased from Sigma Aldrich or Fisher Scientific and used without further purification unless otherwise indicated. Air-free reactions were undertaken using standard Schlenk line and glovebox techniques.

### 2.1 Control Experiments

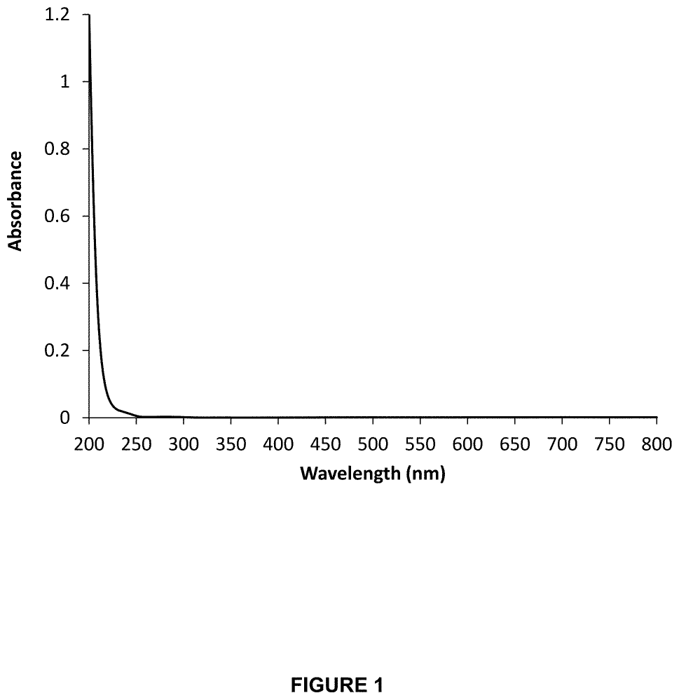

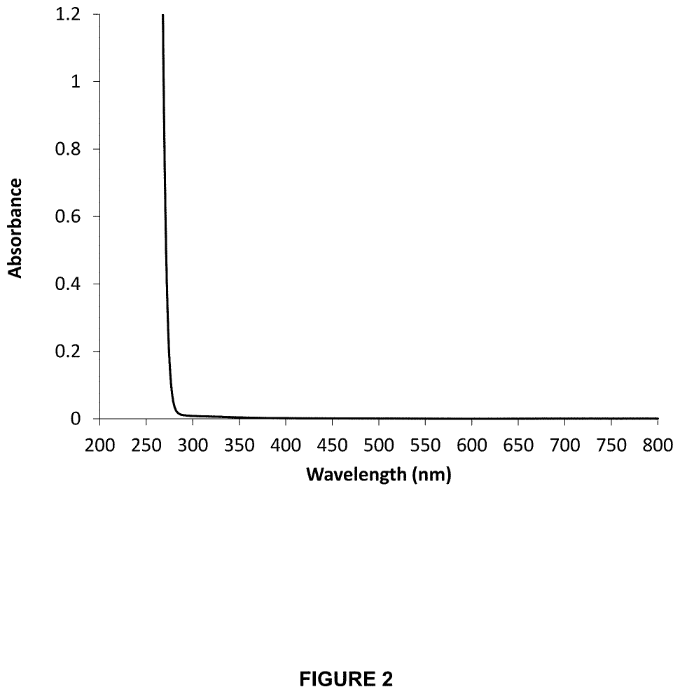

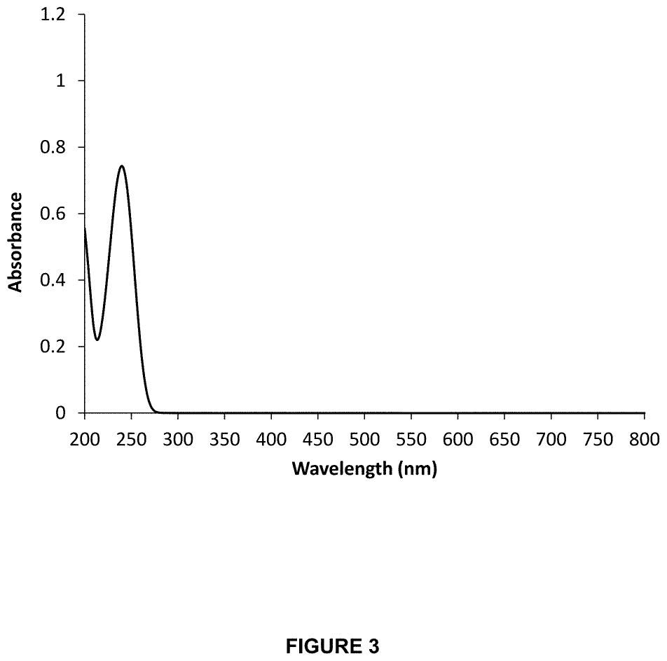

Control UV-Vis absorbance spectra were obtained of methanol, and of solutions of 1,4-diaminobutane (2.0×10⁻³ M) in methanol (FIG. 1) and genipin in methanol (2.0×10⁻³ M, FIG. 2; 8.0×10⁻⁵ M, FIG. 3). Genipin solutions were prepared by addition of 0.10 mmol (23 mg) to 50.0 mL of methanol in a 100 mL round-bottom flask and stirring for about five minutes. A UV-vis spectrum was obtained on a sample of this solution. Subsequently, a 2.0 mL sample of this stock solution was diluted with methanol to a volume of 50.0 mL and UV-vis spectrum obtained on a sample of this solution. In the case of 1,4-diaminobutane, 0.10 mmol (8.8 mg) was added to 50.0 mL of methanol and this solution stirred for five minutes before decanting a sample for UV-vis analysis.

2.2 Reactions of Genipin Solutions with Amines

Reactions were carried out by analogous procedures outlined below, unless otherwise indicated. A single protocol is outlined as follows:

To 0.10 mmol (23 mg) genipin in a 100 mL round-bottom flask was added 40 mL of methanol. 0.10 mmol of an amine was dissolved in 2-3 mL methanol in a vial, and this solution was added to the genipin solution. The vial was rinsed with a further 2-3 mL methanol which was added to the solution and the total volume was made up to 50.0 mL and allowed to stir in the open round bottom flask at room temperature. Prior to measurement of UV-vis absorption at various time points, the solution volume was measured and made up to a consistent 50.0 mL to compensate for evaporation. At no time did total solution volume decrease below 40 mL. After making up to 50.0 mL, the reaction mixture was stirred for an additional five minutes, and an aliquot was decanted into the quartz cuvette for UV-visible analysis.

In instances where dilution of the sample for UV-visible was required, 1.0 mL of the reaction mixture was decanted after making up to 50.0 mL volume, and this sample was diluted to the appropriate volume with methanol and stirred. An aliquot of the dilute solution was decanted into a quartz cuvette and a UV-visible absorbance spectrum obtained. To avoid volume of the reaction mixture exceeding 50.0 mL, the dilute solution was disposed of and the volume lost from the reaction mixture accounted for at the next time of sampling; for example, at the second time point after using 1.0 mL of sample for a dilution, total volume of the reaction mixture was made up to 49.0 mL.

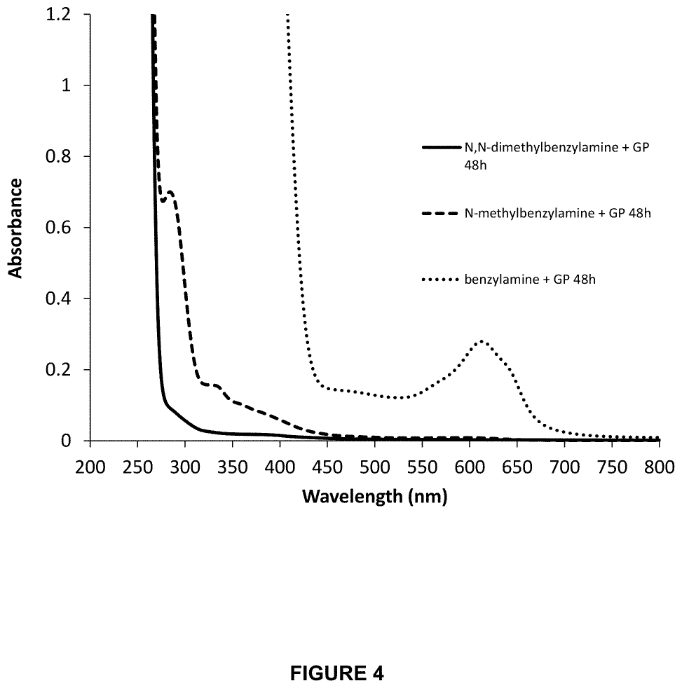

UV-Vis absorbance spectra were run of individual reactions of benzylamine (2.0×10⁻³ M), N-methylbenzylamine (2.0×10⁻³ M), or N,N-dimethylbenzylamine (2.0×10⁻³ M) with genipin (GP) (2.0×10⁻³ M) in methanol after 48 h (FIG. 4). The results showed that genipin is a selective sensor for primary amines over secondary and tertiary amines. The absorbance peak at ˜600 nm correlates to a blue coloration. Many of the amines produced upon decomposition of food are primary amines.

### I. Putrescine (1,4-diaminobutane)

UV-Vis absorbance spectra were run for individual reactions as follows:

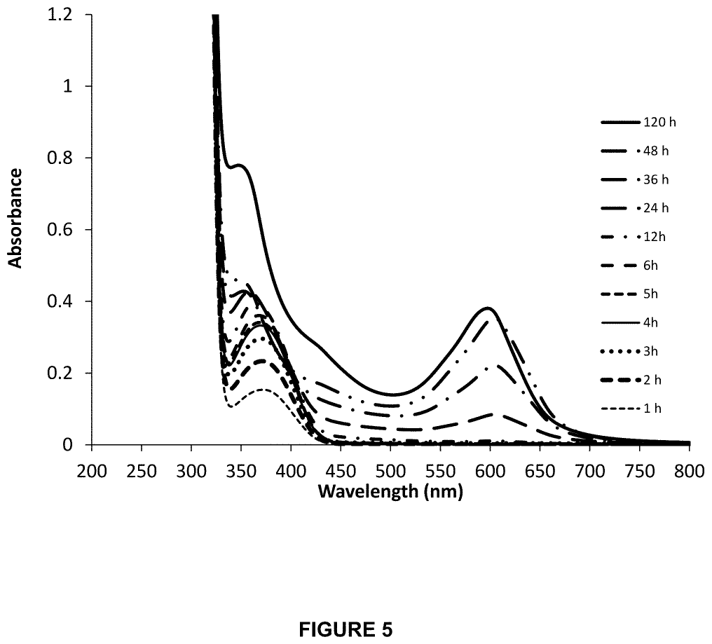

a) A reaction mixture of 1,4-diaminobutane (Putrescine) (2.0×10⁻³ M) with genipin (2.0×10⁻³ M) in 50 ml methanol and monitored over 1-120 h (FIG. 5).

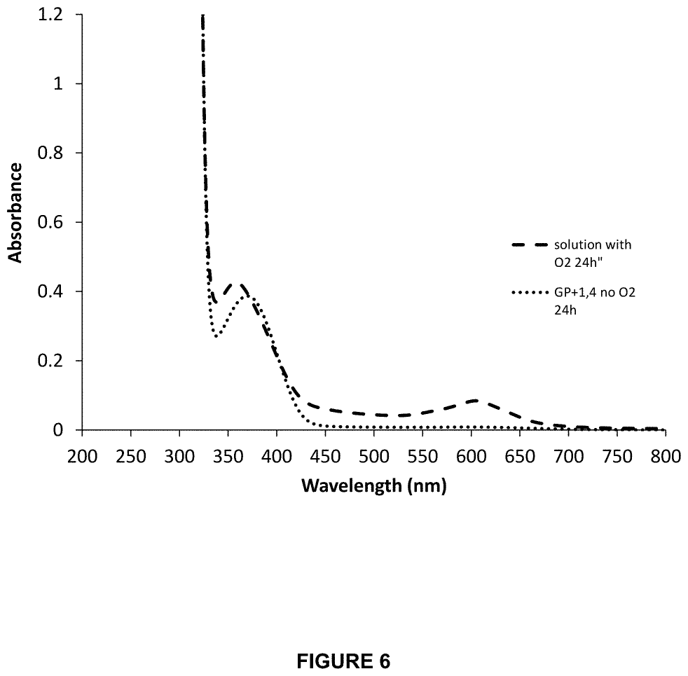

b) A reaction mixture of 1,4-diaminobutane (2.0×10⁻³ M) with genipin (2.0×10⁻³ M) in methanol after 24 h in the presence and absence of oxygen (FIG. 6).

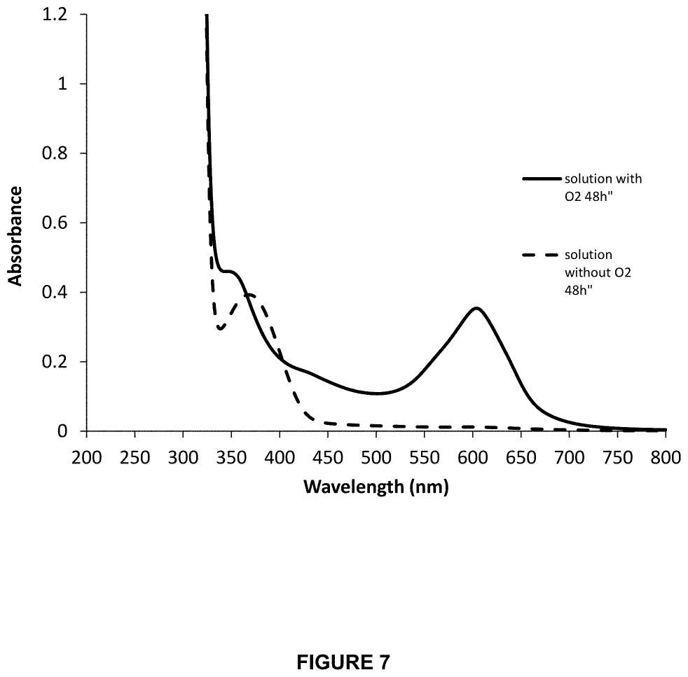

c) A reaction mixture of 1,4-diaminobutane (2.0×10⁻³ M) with genipin (2.0×10⁻³ M) in methanol after 48 h in the presence and absence of oxygen (FIG. 7).

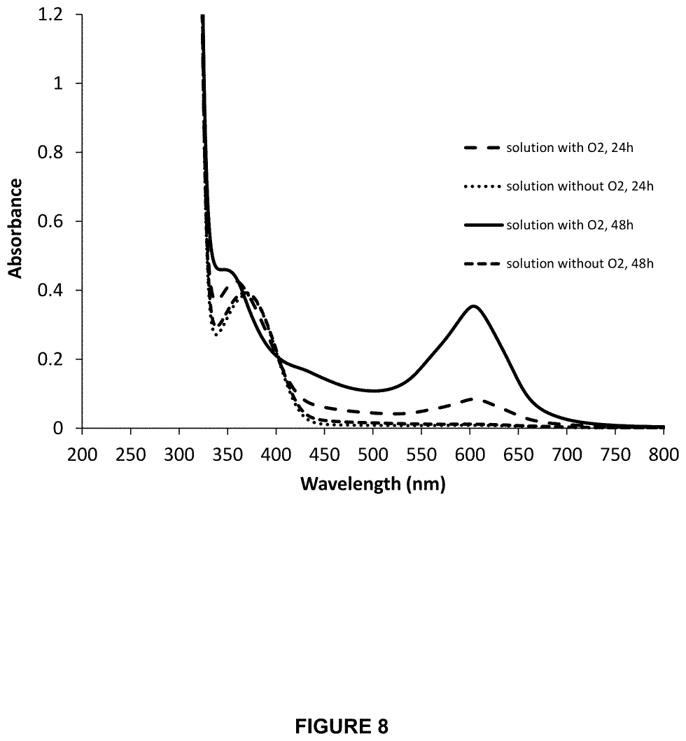

d) A reaction mixture of 1,4-diaminobutane (2.0×10⁻³ M) with genipin (2.0×10⁻³ M) in methanol over 24-48 h in the presence and absence of oxygen (FIG. 8).

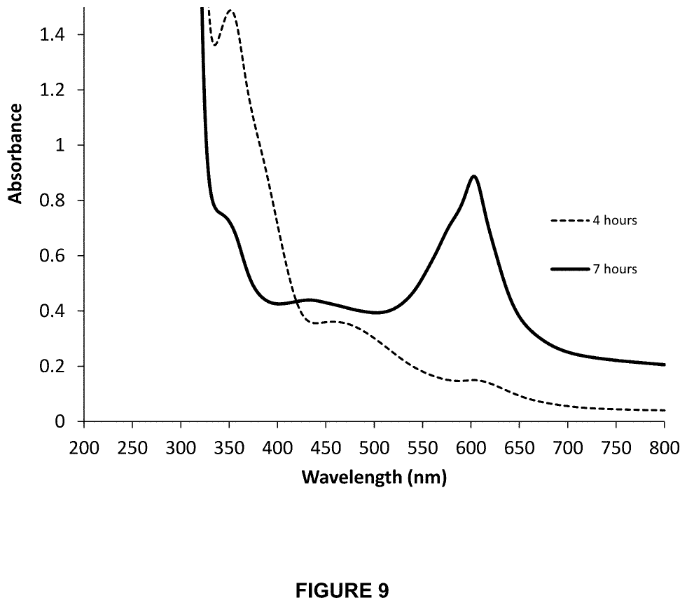

e) A reaction mixture of genipin (0.20M) and 1,4-diaminobutane (0.20 M) in methanol after 4 h and 7 h reaction times and diluted at time of measurement to 2.0×10⁻³M (FIG. 9).

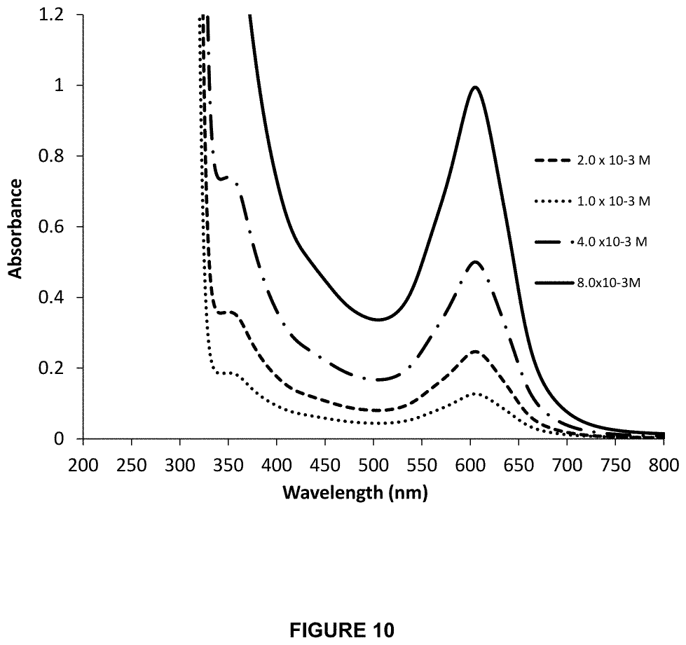

f) A reaction mixture of 2.0×10⁻³ M genipin and 1,4-diaminobutane in methanol after 48 h. Samples were diluted to various concentrations and measured (FIG. 10).

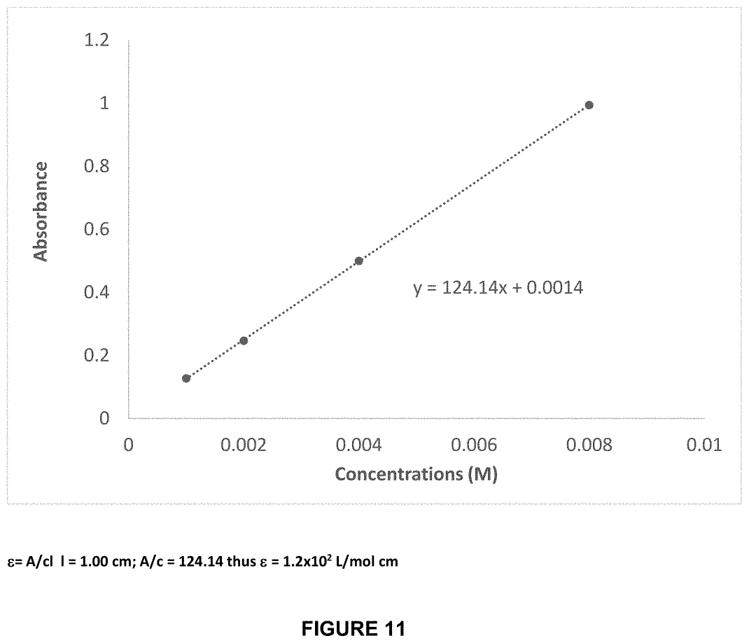

g) λₘₐₓ=605 nm for varying sample concentrations adjusted from a reaction mixture of genipin (2.0×10⁻³ M) and 1,4-diaminobutane (2.0×10⁻³ M) in methanol after 48 h (FIG. 11).

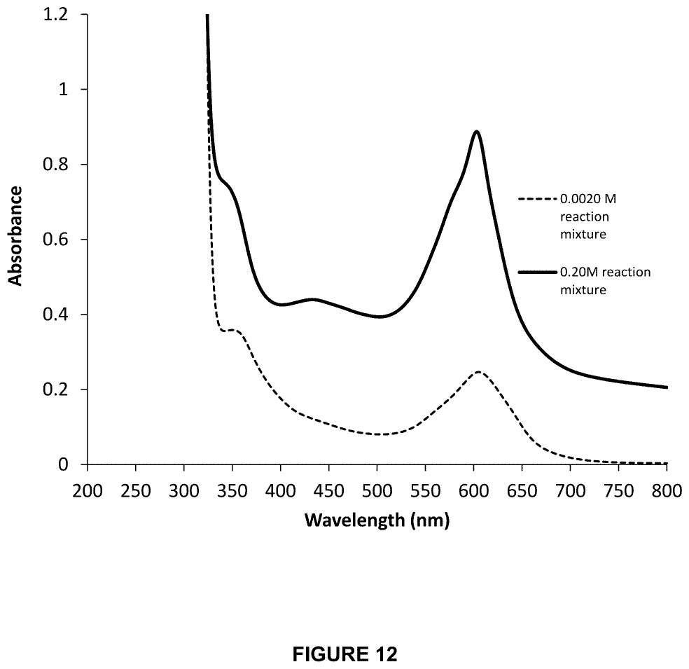

h) A reaction mixture (i) of Genipin (0.20 M) and 1,4-diaminobutane (0.20 M) in methanol after 48 h (diluted at time of measurement to 2.0×10⁻³M) and a reaction mixture (ii) of Genipin (2.0×10⁻³ M) and 1,4-diaminobutane (2.0×10⁻³ M) in methanol after 48 h. (FIG. 12).

A stoichiometric reaction between genipin and 1,4-diaminobutane (putrescine) over time was shown. The colorless material progressed through a red intermediate and finally produced a blue product as the intermediate reacts with oxygen in the solution. In the absence of oxygen, there was no peak at ˜600 nm, an absorbance at ˜380 nm, which corresponded to the red color (FIGS. 6-8) indicating the presence of the amine. Upon addition of oxygen, the absorbance at ˜380 nm decreased and the peak at ˜600 nm increases, indicating that the blue color of the sensor had been triggered in view of oxygen. FIGS. 7 and 8 showed a 48 h timeframe. FIGS. 10-12 showed the effect of different concentrations to extrapolate the molar absorption coefficient and determine the increased absorption at about 605 nm with the increase in amine concentration.

### II. Cadaverine (1,5-diaminopentane)

UV-Vis absorbance spectra were run for individual reactions as follows:

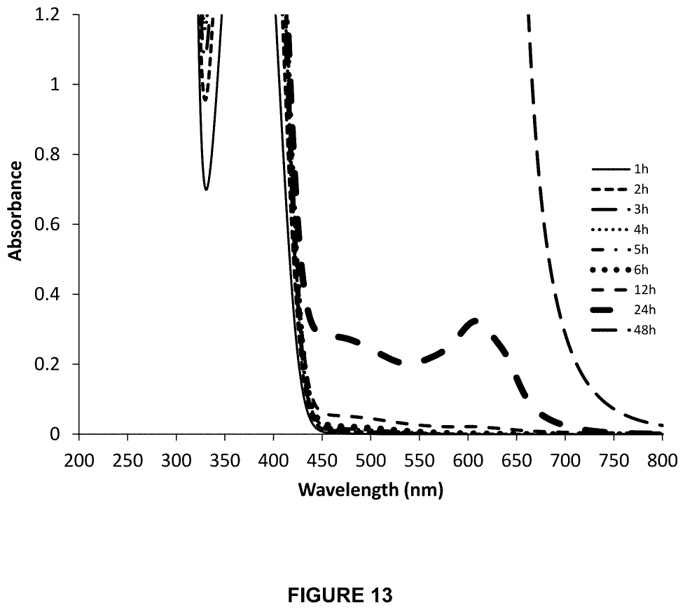

a) UV-vis spectra of reaction mixtures of genipin (2.0×10⁻³ M) and 1,5-diaminopentane (Cadaverine) (2.0×10⁻³ M) in methanol over 1-48 h and diluted 100× at time of measurement (FIG. 13).

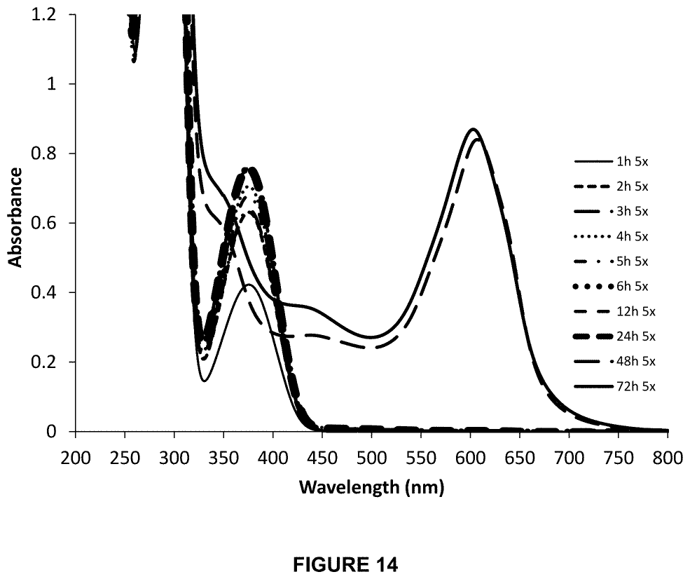

b) UV-vis spectra of reaction mixtures of genipin (2.0×10⁻³ M) and 1,5-diaminopentane (2.0×10⁻³ M) in methanol over 1-48 h diluted 5× at time of measurement (FIG. 14).

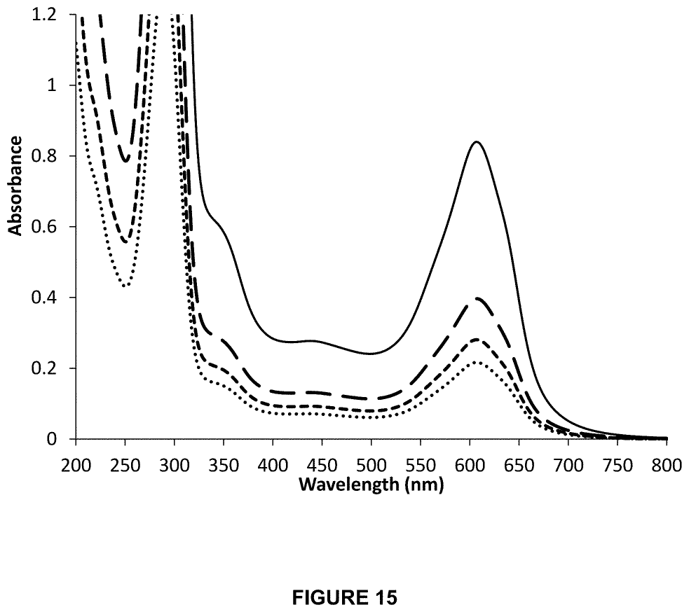

c) UV-vis spectra of reaction mixture of genipin (2.0×10⁻³ M) and 1,5-diaminopentane (2.0×10⁻³ M) in methanol after 48 hours. Samples were diluted to various concentrations and measured (FIG. 15).

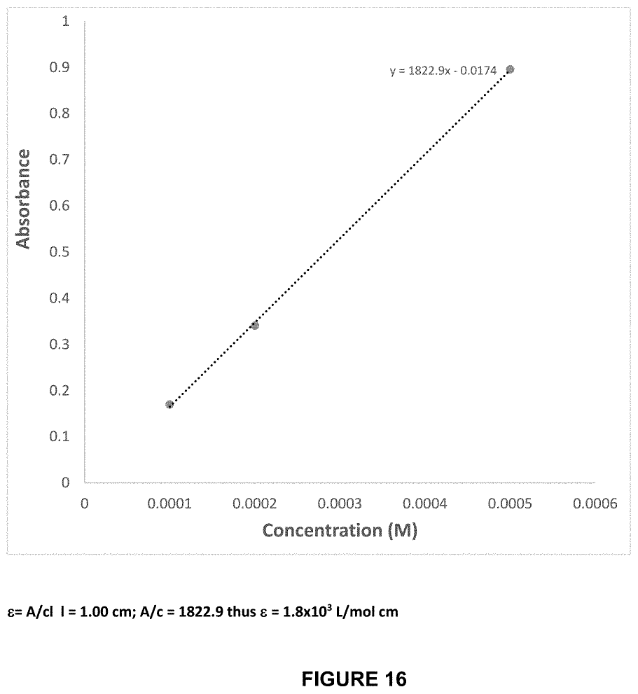

d) Absorbance vs concentration graph showing absorbances at λₘₐₓ=605 nm vs solution concentration for reaction mixtures of genipin and 1,5-diaminopentane reaction mixtures for varying sample concentrations adjusted from a reaction mixture of genipin (2.0×10⁻³ M) and 1,5-diaminopentane (2.0×10⁻³ M) in methanol after 48 h (FIG. 16).

A stoichiometric reaction between genipin and 1,5-diaminopentane was examined. As with the reactions with other amines, colorless solutions progressed through a red intermediate and finally produced a blue color as the intermediate reacted with oxygen in the solution. FIGS. 13 and 14 showed the progress of the reaction over 48 hours, FIG. 15 showed the absorbance after 48 hours at various concentrations, and FIG. 16 graphed the peak absorbances at different concentrations to extrapolate the molar absorption coefficient.

### III. Tyramine (4-(2-aminoethyl)phenol)

UV-Vis absorbance spectra were run for individual reactions as follows:

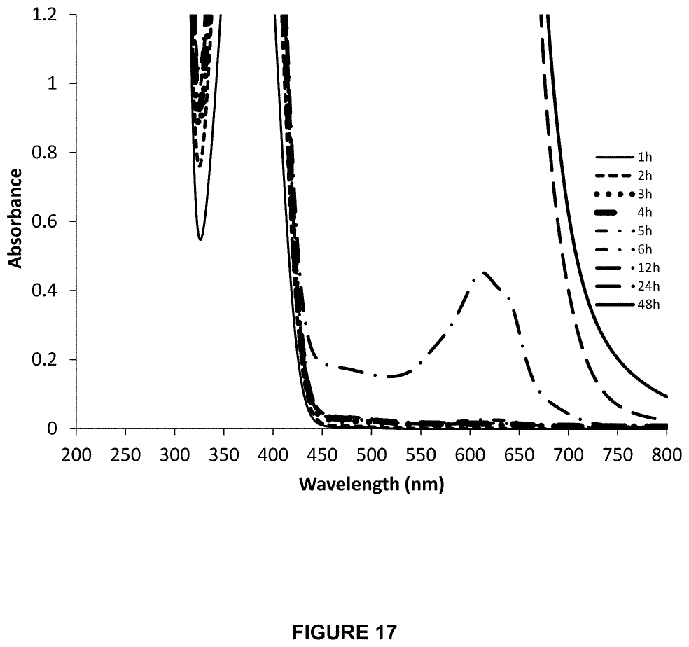

a) Reaction mixtures of genipin (2.0×10⁻³ M) and 4-(2-aminoethyl)phenol (Tyramine) (2.0×10⁻³ M) in methanol over 1-48 h (FIG. 17).

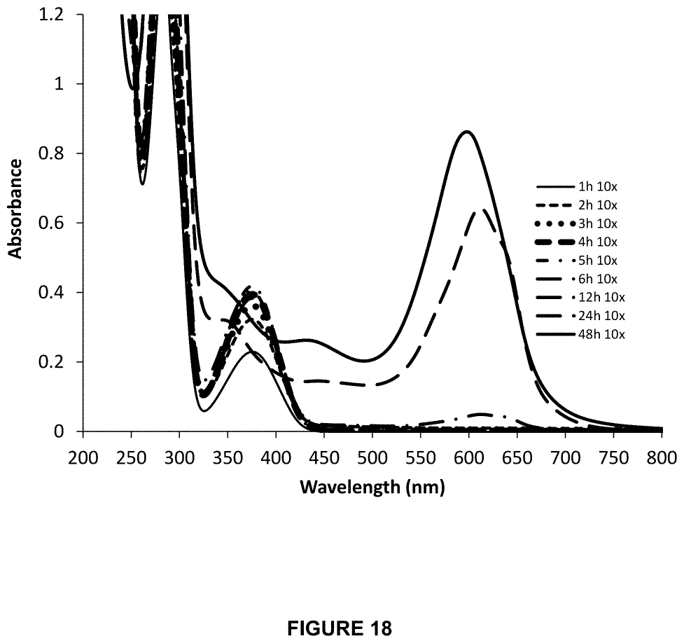

b) Reaction mixtures of genipin (2.0×10⁻³ M) and 4-(2-aminoethyl)phenol (2.0×10⁻³ M) (sample diluted 10×) in methanol over 1-48 h (FIG. 18).

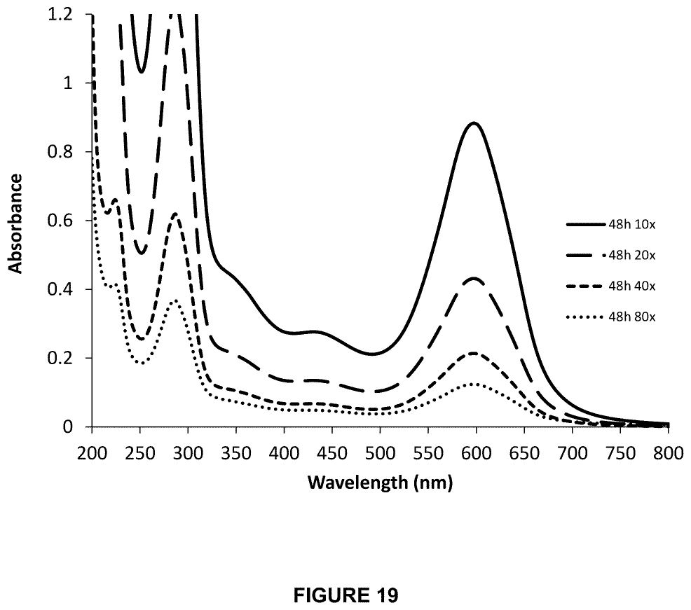

c) Reaction mixtures of genipin (2.0×10⁻³ M) and 4-(2-aminoethyl)phenol (2.0×10⁻³ M) in methanol (FIG. 19).

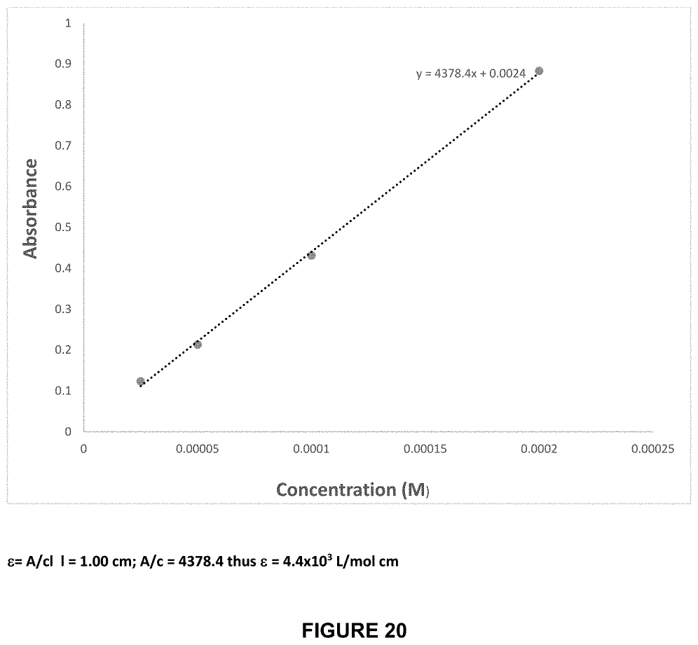

d) λₘₐₓ=605 nm for varying sample concentrations adjusted from a reaction mixture of genipin (2.0×10⁻³ M) and and 4-(2-aminoethyl)phenol (2.0×10⁻³ M) in methanol after 48 h (FIG. 20).

A stoichiometric reaction between genipin and 4-(2-aminoethyl)phenol was examined. As with the reactions with other amines, colorless solutions progressed through a red intermediate and finally produced a blue color as the intermediate reacts with oxygen in the solution. FIGS. 17 and 18 showed the progress of the reaction over 48 hours, FIG. 19 showed the absorbance after 48 hours at various concentrations, and FIG. 20 graphed the peak absorbances at different concentrations to extrapolate the molar absorption coefficient.

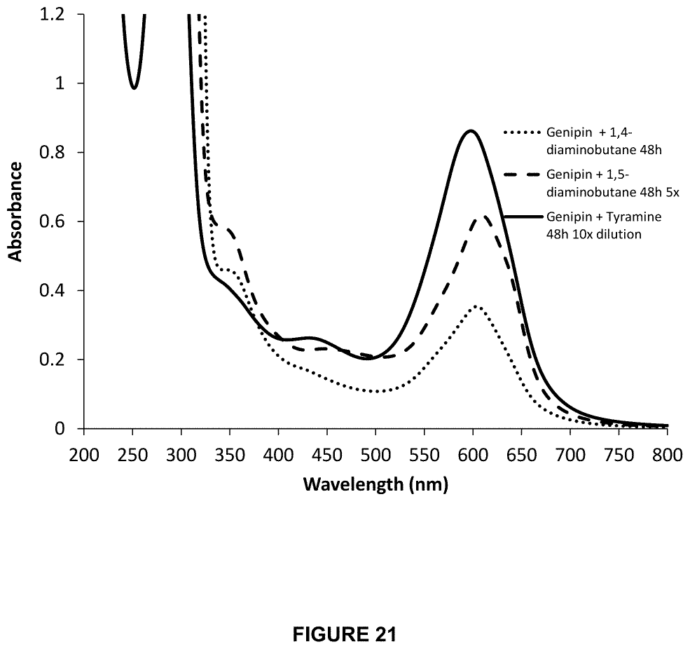

Examples I-III showed that these primary amines trigger a change in color. In the presence of the amine, a red color results and further, in the presence of oxygen, a blue color results in solution. The absorption profiles are summarized for various biogenic amines and genipin in methanol (samples measured at indicated dilutions) (FIG. 21).

### 2.3 Preparation of Genipin-Containing Calcium Alginate Gel Beads (Substrate)

### A) Preparation of Sodium Alginate Gel

50 mg (0.22 mmol) genipin was dissolved in 10 mL of distilled water in a pre-weighed 125 mL Erlenmeyer flask. 200 mg of sodium alginate was then added to the solution and stirred by vigorous rotation of the flasks until a gel consistency was formed. Solutions were further stirred.

### B) Preparation of Calcium Alginate Gel Beads

Approximately 1 mL of gel was taken up in 1 mL disposable syringe without a needle. After dispensing an initial large drop of gel back into the Erlenmeyer flask, the syringe was held about 10 cm above the surface of 25 mL of a 1% mass/volume solution of CaCl₂) in distilled water in a 250 mL Erlenmeyer flask. About 12-15 drops of gel were dispensed into the calcium alginate solution, forming beads of approximately 2-3 mm diameter. This procedure was repeated until 100 beads had been dispensed. The excess CaCl₂) solution was then decanted from the beads, and the beads washed with 10 mL of a 0.5 M acetic acid/acetate buffer solution of pH 4. The 100 gel beads were stored in a refrigerator in a pre-weighed vial.

2.4 Reaction of Genipin Immobilized in Calcium Alginate Gel Beads with Amine Vapors

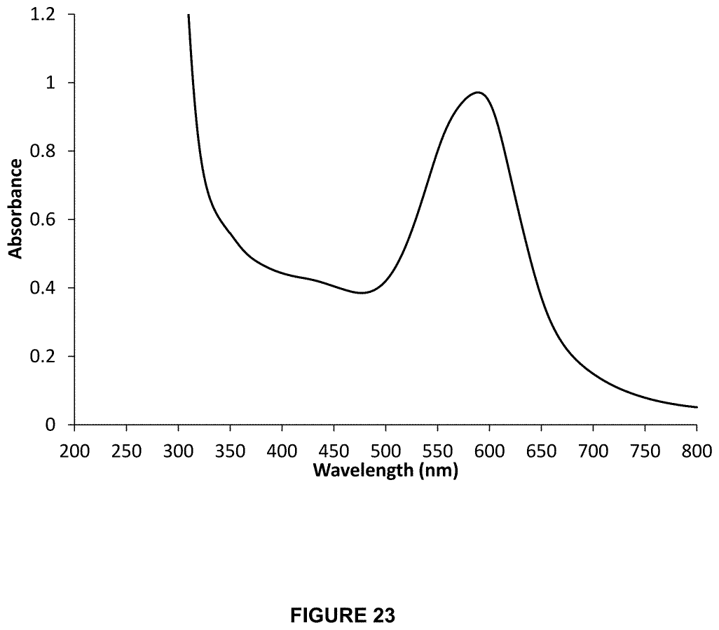

Ten gel beads were placed in an 18 mL vial at either room temperature or in the refrigerator (4° C.). 11-13 mg of amine (1,4-diaminobutane (Putrescine), 1,5-diaminopentane (Cadaverine), 4-(2-aminoethyl)phenol (Tyramine) or benzylamine) was placed in an open small opened plastic insert (or a small opened vial) which was placed in the 18 mL vial, allowing amine vapors to escape to the atmosphere of the 18 mL vial containing the beads. The vial was sealed and monitored visually over 1-48 hours. After 48 hours, the beads were dissolved in about 3.5 mL of a 0.055 M solution of sodium citrate (about 3.5 mM of Genipin). After stirring for about 2 hours, the UV-Visible absorbance spectrum of the solution with putrescine was then measured (FIG. 23).

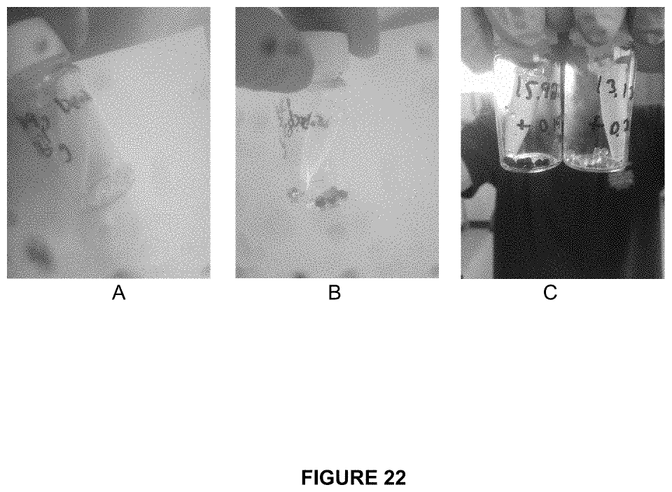

FIG. 22A shows the gel beads at t=0 exposed to 1,4-diaminobutane vapor. FIG. 22B shows the gel beads after 24 hours exposure to 1,4-diaminobutane vapor; FIG. 22C comparison of gel beads in reaction with 1,4-diaminobutane vapor after 48 hours at room temperature (left vial) and in the refrigerator (right vial).

2.5 Study of Genipin Immobilized in Calcium Alginate Gel Beads with Raw Chicken Samples

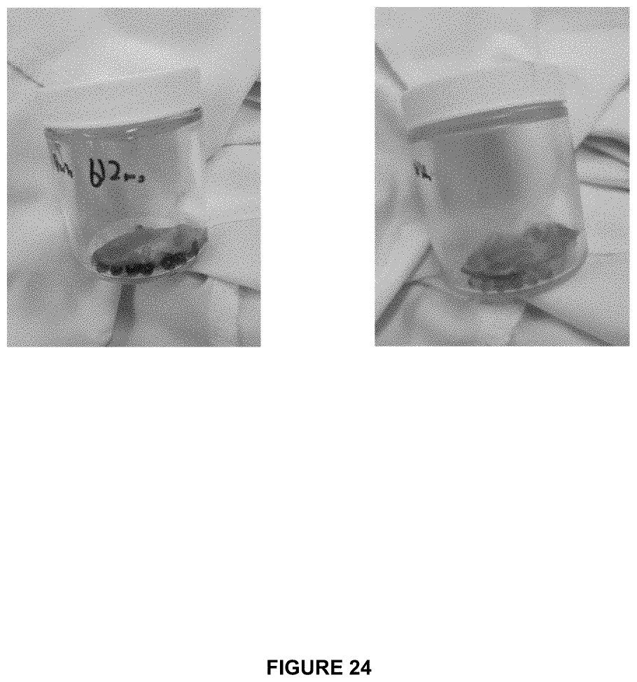

10 genipin-embedded calcium alginate gel beads were placed in each of two 200 mL glass jars using a clean pair of tweezers. A piece of chicken breast, purchased within 48 hours from a commercial grocery store, was cut into about 5 g to 6 g portions and one portion was placed in each glass jar using a separate, clean pair of tweezers, such that the gel beads were in contact with the surface of the raw chicken. The glass jars were sealed with a plastic screw cap. One was left at room temperature, while one was refrigerated at about 4° C. Both samples were monitored visually and photographed each hour from 1 hour to 6 hours, and then photographed again after 24 hours. This procedure was repeated three times with fresh beads and chicken. FIG. 24 shows Genipin beads placed next to raw chicken after 2 hours at room temperature (left) and two hours in the refrigerator at 4° C. (right).

3.1 Preparation of Genipic Acid from Geniposidic Acid

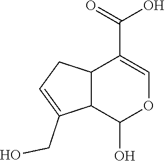 [SMILES: O=C(O)C1=COC(O)C2C(CO)=CCC12]

To a 30 mL solution of geniposidic acid (0.19 g, 50 mmol) in 0.1M CH₃COOH/CH₃COONa buffer was added 390 mg of 1.3 units/mg cellulase from Aspergillus niger. The solution was stirred in a 200 mL round bottom flash at 50° C. for eighteen hours. It was then cooled, extracted with 3×30 mL ethyl acetate and the combined ethyl acetate extracts dried over MgSO₄ and filtered through Celite. Solvent was removed from the resulting pale golden solution to yield a colorless oil. A sample of the oil dissolved in deuterated methanol exhibited a ¹H NMR spectrum showing genipic acid in >90% purity.

¹H NMR (300 MHz, CD₃OD): 7.55 (s, 1H); 5.84 (s, 1H); 4.81 (d, 1H, ³J_{HH}=8.4 Hz); 4.27 (m, 2H); 3.16 (dd, 1H, ³J_{HH}=16.9 Hz, ³J_{HH}=8.4 Hz); 2.85 (m, 1H); 2.50 (t, 1H, ³J_{HH}=8.0 Hz); 2.06 (1H, m)

Genipic acid was found to also react with primary amines to produce a visible color change (e.g. blue color).

1. A colorimetric sensor for detecting an analyte, wherein the analyte comprises an amine, the sensor comprising genipin, a genipin derivative, a salt thereof, hydrate thereof, solvate thereof, tautomer thereof, optical isomer thereof, or a combination thereof.

2. The sensor of claim 1, wherein there is a visible color change.
wherein the amine comprises a primary amine; and/or
wherein the amine is selected from ammonia, biogenic amines, amino acids, amides, polypeptides, proteins, substituted and unsubstituted alkyl amines, substituted and unsubstituted aromatic amines, substituted and unsubstituted heteroaromatic amines, or a combination thereof.

3-4. (canceled)

5. The sensor of claim 1, wherein the amine is selected from benzyl amine, 4-(2-aminoethyl)phenol, N-methylbenzylamine, N,N-dimethylbenzylamine, 1,4 diaminobutane, 1,5-diaminopentane, or a combination thereof; optionally, the amine is selected from 4-(2-aminoethyl)phenol, 1,4 diaminobutane, 1,5-diaminopentane, or a combination thereof.

6. (canceled)

7. The sensor of, claim 1, wherein the amine is vaporous; optionally, the amine has a high vapor pressure is a gaseous amine.

8-9. (canceled)

10. The sensor of claim 1, wherein the genipin, the genipin derivative, the salt thereof, hydrate thereof, solvate thereof, tautomer thereof, optical isomer thereof, or the combination thereof undergoes a visible color change upon exposure to the amine; optionally, the genipin undergoes a visible color change upon exposure to the amine or wherein the genipin derivative undergoes a visible color change upon exposure to the amine.

11-12. (canceled)

13. The sensor of claim 12, wherein the genipin derivative is: 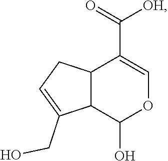 [SMILES: O=C(O)C1=COC(O)C2C(CO)=CCC12]
a salt thereof, hydrate thereof, solvate thereof, tautomer thereof, optical isomer thereof, or a combination thereof.

14. The sensor of claim 10, wherein the visible color change is a color that absorbs in a wavelength range of about 375 nm to about 490 nm; or wherein the visible color change is a reddish color.

15. (canceled)

16. The sensor of claim 1 wherein the sensor comprises genipin.

17. The sensor of claim 1, wherein the analyte further comprises an oxidizing agent; optionally, the oxidizing agent is selected from oxygen, ozone, nitric oxide, nitrous oxide, hydrogen peroxide, or a combination thereof; or the oxidizing agent is oxygen.

18-19. (canceled)

20. The sensor of claim 17, wherein the genipin, the genipin derivative, the salt thereof, hydrate thereof, solvate thereof, tautomer thereof, optical isomer thereof, or the combination thereof undergoes a further visible color change upon exposure to the oxidizing agent.
wherein the genipin undergoes a further visible color change upon exposure to the oxidizing agent; or
wherein the genipin derivative undergoes a further visible color change upon exposure to the oxidizing agent.

21-22. (canceled)

23. The sensor of claim 20, wherein the visible color change is a color that absorbs in a wavelength range of about 550 nm to about 650 nm.
wherein the visible color change is a blue/violet color.

24. (canceled)

25. The sensor of claim 1, further comprising a solvent; the solvent is selected whereby the genipin, the genipin derivative, the salt thereof, hydrate thereof, solvate thereof, tautomer thereof, optical isomer thereof, or the combination thereof is sufficiently soluble such that the colorimetric sensor is capable of changing color in the presence of the amine;
wherein the solvent is selected from water, alcohols, or a combination thereof; or
wherein the solvent is a vegetable oil, a natural oil, an essential oil, or a combination thereof.

26-28. (canceled)

29. The sensor of claim 1, further comprising a thickening agent.

30. The sensor of claim 1, wherein the sensor is a gel:
wherein the sensor is multiple particulates; and/or
wherein the sensor is selected from powder, granules, tablets, pellets, beads, mini-tabs, spherules, beadlets, microcapsules, milli-spheres, nano-capsules, micro-spheres, capsules, or a combination thereof, optionally, the capsules comprises a semi-permeable coating or semi-permeable encapsulation.

31-33. (canceled)

34. The sensor of claim 1, further comprising a substrate; optionally, the genipin, the genipin derivative, the salt thereof, hydrate thereof, solvate thereof, tautomer thereof, optical isomer thereof, or the combination thereof is in the form of a coating on the substrate, intermixed with the substrate to form a matrix, impregnated in the substrate, at least a portion covalently bound to the substrate, non-covalently bound to the substrate, or a combination thereof; or
the genipin, the genipin derivative, the salt thereof, hydrate thereof, solvate thereof, tautomer thereof, optical isomer thereof, or the combination thereof is non-covalently bound to the substrate; and/or
the substrate is selected from a woven or non-woven material, a polymer, or a combination thereof; optionally, the substrate is selected from paper, cotton, fabric, sponge, foam, glass and ceramic fibre paper, nylon, rayon, a matrix, bead, a film-forming or fiber-forming polymer, or a combination thereof; the substrate is polymeric; the substrate is polymeric packaging; and/or the substrate is a gel substrate; optionally, the gel substrate is bead(s) or a film.

35-42. (canceled)

43. The sensor of claim 34, wherein the gel substrate comprises a carbohydrate polymer; optionally, the carbohydrate polymer comprises an anionic polysaccharide, an alkali and/or alkaline earth metal alginate.

44-45. (canceled)

46. The sensor of claim 43, wherein the sensor comprises beads, wherein the beads comprise genipin and the alginate; optionally, the beads are in a container such as a pouch, sachet or vial that is permeable or semi-permeable.

47. (canceled)

48. The sensor of claim 1, further comprising an additive to control the color change of genipin, a genipin derivative, a salt thereof, hydrate thereof, solvate thereof, tautomer thereof, optical isomer thereof, or a combination thereof; optionally, the additive is a regulator that inhibits or enhances the reaction of the analyte with genipin, a genipin derivative, a salt thereof, hydrate thereof, solvate thereof, tautomer thereof, optical isomer thereof, or a combination thereof.

49. (canceled)

50. A container comprising the colorimetric sensor of claim 1; optionally, the container is selected from a pouch, sachet, or vial and/or the container is permeable or semi-permeable.

51-52. (canceled)

53. A system comprising the colorimetric sensor of claim 1.

54. A device comprising the colorimetric sensor of claim 1.

55. (canceled)

56. A method for detecting the presence of the amine, the method comprising exposing the sensor of claim 1 to the amine, the method comprising exposing the sensor of claim 1 to the amine; optionally, the amine is released from food spoilage and/or the amine is released in an industrial facility.

57-58. (canceled)
